(19) United States
(12) Patent Application Publication
(10) Pub. No.: US 2025/0302533 A1
(43) Pub. Date: Oct. 2, 2025

(54) ORAL IMAGE PROCESSING DEVICE AND ORAL IMAGE PROCESSING METHOD

(71) Applicant: MEDIT CORP., Seoul (KR)

(72) Inventor: Sung Hoon LEE, Seoul (KR)

(73) Assignee: MEDIT CORP., Seoul (KR)

(21) Appl. No.: 18/698,526

(22) PCT Filed: Oct. 10, 2022
(86) PCT No.: PCT/KR2022/015232

§ 371 Date: Oct. 10, 2024

(30) Foreign Application Priority Data
Oct. 8, 2021 (KR) 10-2021-0134443

(51) Int. Cl. A61B 34/10; A61C 9/00; G06T 7/00

(52) U.S. Cl. CPC A61B 34/10; A61C 9/0053; G06T 7/0012; A61B 2034/102; G06T 2207/30036

(57) ABSTRACT

Provided are an intraoral image processing apparatus and an intraoral image processing method. In an embodiment, the intraoral image processing method may include obtaining an intraoral image including a tooth, setting an insertion direction of a prosthesis corresponding to the tooth, obtaining an undercut area included in the tooth based on the set insertion direction and the intraoral image, and compensating the undercut area based on the set insertion direction. In an embodiment, the intraoral image processing method may include obtaining an intraoral image including a prosthesis target tooth, generating a prosthesis by applying a preset reference insertion direction to the intraoral image, setting an insertion direction of the prosthesis based on a shape of the prosthesis target tooth, and changing an inner shape of the prosthesis based on the set insertion direction.

### TECHNICAL FIELD

Embodiments relate to an intraoral image processing apparatus and an intraoral image processing method, and more particularly, to an intraoral image processing apparatus and method for preventing the occurrence of an undercut or compensating an undercut.

### BACKGROUND ART

Dental computer-aided design/computer-aided manufacturing (CAD/CAM) technology is widely used in dental treatment, particularly prosthetic treatment or the like. The most important thing in dental treatment using CAD/CAM is to obtain precise three-dimensional data about the shape of an object such as a patient's tooth, gum, or jawbone. When three-dimensional data obtained from an object is used to perform dental treatment, accurate calculation may be performed by a computer. For example, methods such as computed tomography (CT), magnetic resonance imaging (MRI), and optical scanning may be used to obtain three-dimensional data of an object in dental treatment using dental CAD/CAM.

When an undercut that is a tooth region between a height of contour and a gingiva exists in a tooth, it may be difficult to perform dental treatment using prosthesis or the like. Thus, it may be necessary to identify whether an undercut exists in a tooth in dental treatment using dental CAD/CAM.

### DISCLOSURE

### Technical Problem

Embodiments provide an intraoral image processing method for compensating an undercut area based on an insertion direction of a prosthesis and an apparatus for performing an operation according thereto. Also, embodiments provide an intraoral image processing method for changing an inner shape of a prosthesis based on a set insertion direction of the prosthesis and an apparatus for performing an operation according thereto.

### Technical Solution

An intraoral image processing method according to an embodiment may include obtaining an intraoral image including a tooth. The intraoral image processing method may include setting an insertion direction of a prosthesis corresponding to the tooth. The intraoral image processing method may include obtaining an undercut area included in the tooth based on the set insertion direction and the intraoral image. The intraoral image processing method may include compensating the undercut area based on the set insertion direction.

In an embodiment, the tooth may include a plurality of points along a surface shape of the tooth. The setting of the insertion direction may include setting the insertion direction based on a shape of the tooth. The insertion direction may be set based on at least one of the shape of the tooth, a shape of adjacent teeth around the tooth, an arrangement between the tooth and the adjacent teeth, or an average normal direction of the plurality of points included in the tooth.

In an embodiment, the obtaining of the undercut area may include providing a virtual line in a direction parallel to the insertion direction from each of the plurality of points. The obtaining of the undercut area may include obtaining, as the undercut area, an area of the tooth including at least one point providing a virtual line intersecting the tooth.

In an embodiment, the at least one point included in the undercut area may be defined as a first reference point, at least one point meeting the virtual line provided from the first reference point may be defined as a second reference point, and a center of an extension line passing through the first reference point and the second reference point may be defined as a third reference point. The compensating of the undercut area may include obtaining a center point located at a center of the tooth. The compensating of the undercut area may include obtaining a reference direction from the center point toward the third reference point. The compensating of the undercut area may include moving the first reference point in a same direction as the reference direction to compensate the undercut area.

In an embodiment, the compensating of the undercut area may include, before the moving of the first reference point, moving the center point to be on a same line as the first reference point.

In an embodiment, the compensating of the undercut area may include obtaining a first reference direction from the center point toward the first reference point. The compensating of the undercut area may include calculating a normal direction from the first reference point toward an outside of the tooth. The compensating of the undercut area may include, when an angle between a vector with the first reference direction and a vector with the normal direction is greater than 90 degrees, moving the first reference point in a direction opposite to the first reference direction to compensate the undercut area.

In an embodiment, the compensating of the undercut area may include moving at least one point included in the undercut area in a direction perpendicular to the insertion direction and toward an outside of the tooth, to compensate the undercut area.

In an embodiment, the compensating of the undercut area may be repeated until the virtual line provided from each of the plurality of points does not intersect the tooth in the obtaining of the undercut area.

In an embodiment, the intraoral image processing method may include, after the compensating of the undercut area, simplifying and smoothing the compensated undercut area.

An intraoral image processing apparatus according to an embodiment may include a memory storing at least one instruction and at least one processor configured to execute the at least one instruction stored in the memory. The at least one processor may be configured to obtain an intraoral image including a tooth. The at least one processor may be configured to set an insertion direction of a prosthesis corresponding to the tooth. The at least one processor may be configured to obtain an undercut area included in the tooth based on the set insertion direction and the intraoral image. The at least one processor may be configured to compensate the undercut area based on the set insertion direction.

An intraoral image processing method according to an embodiment may include obtaining an intraoral image including a prosthesis target tooth. The intraoral image processing method may include generating a prosthesis by applying a preset reference insertion direction to the intraoral image. The intraoral image processing method may include setting an insertion direction of the prosthesis based on a shape of the prosthesis target tooth. The intraoral image processing method may include changing an inner shape of the prosthesis based on the set insertion direction.

In an embodiment, the changing of the inner shape of the prosthesis may include generating a virtual inner shape by applying the set insertion direction to the intraoral image. The changing of the inner shape of the prosthesis may include changing the inner shape of the prosthesis by comparing the inner shape of the prosthesis with the virtual inner shape. In an embodiment, a margin line of the inner shape of the prosthesis and a margin line of the virtual inner shape may be same as each other, and the inner shape of the prosthesis may have a shape extending from the margin line in the reference insertion direction. The virtual inner shape may have a shape extending from the margin line in the set insertion direction.

In an embodiment, the inner shape of the prosthesis may include a plurality of points along a surface of the inner shape. The changing of the inner shape of the prosthesis may include providing a virtual line in a direction opposite to a normal from each of the plurality of points. The changing of the inner shape of the prosthesis may include changing a shape of an area in the inner shape of the prosthesis, which includes at least one point providing a virtual line intersecting the virtual inner shape, to correspond to the virtual inner shape.

In an embodiment, the intraoral image processing method may include, after the changing of the inner shape of the prosthesis, simplifying and smoothing the changed inner shape of the prosthesis.

An intraoral image processing apparatus according to an embodiment may include a memory storing at least one instruction and at least one processor configured to execute the at least one instruction stored in the memory. The at least one processor may be configured to obtain an intraoral image including a prosthesis target tooth. The at least one processor may be configured to generate a prosthesis and an inner shape of the prosthesis by applying a preset reference insertion direction to the intraoral image. The at least one processor may be configured to set an insertion direction of the prosthesis based on a shape of the prosthesis target tooth. The at least one processor may be configured to change the inner shape of the prosthesis based on the set insertion direction.

### Advantageous Effects

The intraoral image processing apparatus and the intraoral image processing method according to embodiments may compensate an undercut area included in a tooth. Accordingly, an intraoral image including a tooth with a compensated undercut area may be obtained.

The intraoral image processing apparatus and the intraoral image processing method according to embodiments may change an inner shape of a prosthesis based on a set insertion direction. Accordingly, the inner shape of the prosthesis may be changed based on the set insertion direction and thus an intraoral image that does not include an undercut area may be obtained.

### DESCRIPTION OF DRAWINGS

The present disclosure may be easily understood through the following detailed description and the accompanying drawings, in which reference numerals refer to structural elements.

FIG. 1 is a diagram for describing an intraoral image processing system according to an embodiment of the present disclosure.

FIG. 2 is a block diagram for describing an intraoral image processing system according to an embodiment of the present disclosure.

FIG. 3 is a diagram for describing an undercut area according to an embodiment of the present disclosure.

FIG. 4 is a flowchart for describing an intraoral image processing method according to an embodiment of the present disclosure.

FIG. 5 is a diagram for describing an undercut area and a prosthesis according to an embodiment of the present disclosure.

FIG. 6A is a diagram for describing an operation of compensating an undercut area according to an embodiment of the present disclosure.

FIG. 6B is a diagram for describing an operation of compensating an undercut area according to an embodiment of the present disclosure.

FIG. 6C is a diagram for describing an operation of compensating an undercut area according to an embodiment of the present disclosure.

FIG. 7 is a diagram for describing an intraoral image processing apparatus and method according to an embodiment of the present disclosure.

FIG. 8 is a flowchart for describing an intraoral image processing method according to an embodiment of the present disclosure.

FIG. 9 is a diagram for describing a reference direction, a prosthesis, and an inner shape of the prosthesis according to an embodiment of the present disclosure.

FIG. 10A is a diagram for describing a set direction and a virtual inner shape according to an embodiment of the present disclosure.

FIG. 10B is a diagram for describing an operation of changing an inner shape of a prosthesis according to an embodiment of the present disclosure.

FIG. 10C is a diagram for describing an intraoral image processing apparatus and method according to an embodiment of the present disclosure.

### MODE FOR INVENTION

The specification clarifies the scope of the present disclosure and describes the principle of the present disclosure and embodiments so that those of ordinary skill in the art may implement the present disclosure. The embodiments may be implemented in various forms.

Throughout the specification, like reference numerals will denote like elements. The specification may not describe all elements of the embodiments, and general descriptions in the art to which the present disclosure belongs or redundant descriptions between the embodiments will be omitted for conciseness. The term ‘unit’ (or part or portion) used herein may be implemented as software or hardware, and depending on the embodiments, a plurality of ‘units’ may be implemented as one element (or unit) or one ‘unit’ may include a plurality of elements. Hereinafter, the operation principle and embodiments of the present disclosure will be described with reference to the accompanying drawings.

Herein, an image may include an image representing at least one tooth or an oral cavity including at least one tooth (hereinafter referred to as “intraoral image”).

Also, herein, the image may be a two-dimensional image of an object or a three-dimensional model or a three-dimensional image stereoscopically representing an object. Also, herein, the image may refer to data required to two-dimensionally or three-dimensionally represent an object, for example, raw data obtained from at least one image sensor. Particularly, the raw data may be data obtained to generate an image, and when an object is scanned by using a three-dimensional scanner, the raw data may be data (e.g., two-dimensional data) obtained from at least one image sensor included in the three-dimensional scanner.

Herein, the ‘object’ may include a tooth, a gingiva, at least some area of the oral cavity, and/or an artificial structure insertable into the oral cavity (e.g., an orthodontic device, an implant, an artificial tooth, or an orthodontic aid tool inserted into the oral cavity). Here, the orthodontic device may include at least one of a bracket, an attachment, an orthodontic screw, a lingual orthodontic device, and a removable orthodontic retainer.

Herein, the ‘intraoral image’ may include various polygonal meshes. For example, when two-dimensional data is obtained by using an intraoral scanner, a data processing apparatus may calculate the coordinates of a plurality of illuminated surface points by using a triangulation method. By using an intraoral scanner to perform scanning while moving along the surface of an object, the coordinates of the surface points may be accumulated as the amount of scan data increases. As a result of the image obtainment, a point cloud of vertexes may be identified to represent the extent of the surface. The points in the point cloud may represent actually measured points on the three-dimensional surface of the object. The surface structure may be approximated by forming a polygonal mesh in which adjacent vertexes of the point cloud are connected by a line segment. The polygonal mesh may be variously determined, such as a triangular mesh, a square mesh, or a pentagonal mesh. The relationship between the polygon of a mesh model and an adjacent polygon may be used to extract features of the tooth boundary, such as curvature, minimum curvature, edge, and spatial relationship.

Hereinafter, embodiments will be described in detail with reference to the accompanying drawings.

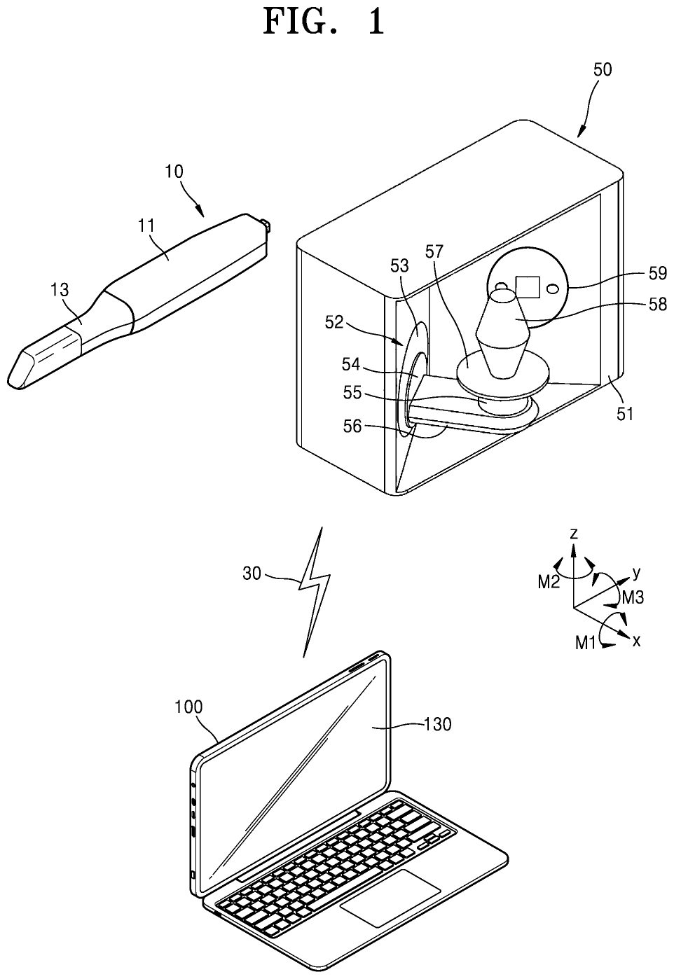

FIG. 1 is a diagram for describing an intraoral image processing system according to an embodiment of the present disclosure.

Referring to FIG. 1, the intraoral image processing system may include three-dimensional scanners 10 and 50 and an intraoral image processing apparatus 100. The three-dimensional scanners 10 and 50 and the intraoral image processing apparatus 100 may communicate with each other through a communication network 30.

The three-dimensional scanners 10 and 50 may be devices scanning an object and may be medical devices obtaining an image of an object. In an embodiment, the object may include any object or body to be scanned by the three-dimensional scanners 10 and 50. In an embodiment, the object may include at least one of an oral cavity, an artificial structure, or a plaster model modeled after an oral cavity or an artificial structure.

The three-dimensional scanners 10 and 50 may include at least one of an intraoral scanner 10 and a table scanner 50.

In an embodiment, the three-dimensional scanners 10 and 50 may include the intraoral scanner 10. The intraoral scanner 10 may be a handheld type scanner that scans an oral cavity while a user holds and moves the scanner in a hand. The intraoral scanner 10 may obtain an image of an oral cavity including at least one tooth by being inserted into the oral cavity and scanning teeth in a non-contact manner. Also, the intraoral scanner 10 may have a form capable of being inserted into and withdrawn from the oral cavity and may scan the inside of the patient's oral cavity by using at least one image sensor (e.g., an optical camera).

The intraoral scanner 10 may include a main body 11 and a tip 13. The main body 11 may include a light irradiating unit (not illustrated) for projecting light and a camera (not illustrated) for obtaining an image by photographing an object.

The tip 13 may be a portion inserted into the oral cavity and may be detachably mounted on the main body 11. The tip 13 may include an optical path changing unit to direct the light emitted from the main body 11 to the object and to direct the light received from the object to the main body 11.

In order to image the surface of at least one of a tooth and a gingiva inside the oral cavity and an artificial structure insertable into the oral cavity (e.g., an orthodontic device including brackets and wires, an implant, an artificial tooth, or an orthodontic assistance tool inserted into the oral cavity), the intraoral scanner 10 may obtain surface information about the object as raw data.

In an embodiment, based on the obtained raw data, the intraoral scanner 10 may image a representation of at least one of a tooth and a gingiva inside the oral cavity and an artificial structure insertable into the oral cavity to obtain a two-dimensional intraoral image two-dimensionally representing the oral cavity.

In an embodiment, the three-dimensional scanners 10 and 50 may include the table scanner 50. The table scanner 50 may be a scanner for obtaining surface information about an object 58 as raw data by scanning the object 58 by using the rotation of a table 57. The table scanner 50 may scan the surface of the object 58 such as a plaster model or an impression model modeled after the oral cavity, an artificial structure insertable into the oral cavity, or a plaster model or an impression model modeled after an artificial structure. In an embodiment, based on the obtained raw data, the table scanner 50 may image a representation of at least one of a tooth and a gingiva inside the oral cavity and an artificial structure insertable into the oral cavity to obtain a two-dimensional intraoral image two-dimensionally representing the oral cavity.

The table scanner 50 may include an internal space formed by being recessed toward the inside of a housing 51. A moving unit 52, on which the object 58 may be mounted and which may move the object 58, may be formed on the side surface of the internal space. The moving unit 52 may move vertically in a z-axis direction. The moving unit 52 may include a fixed base 53 connected to a first rotating unit 54, the first rotating unit 54 rotatable in a first rotation direction M1 with a point on the fixed base 53 as a central axis, for example, with an x axis as a central axis, and a beam portion 56 connected to the first rotating unit 54 and formed to protrude from the first rotating unit 54. The beam portion 56 may extend or shorten in an x-axis direction.

A second rotating unit 115 with a cylindrical shape that may rotate in a second rotation direction M2 with a z axis as a rotation axis may be coupled to the other end of the beam portion 56. The table 57 rotating together with the second rotating unit 55 may be formed on one surface of the second rotating unit 55.

An optical unit 59 may be formed in the internal space. The optical unit 59 may include a light irradiating unit for projecting patterned light onto the object 58 and at least one camera for receiving the light reflected from the object 58 to obtain a plurality of two-dimensional frames. The optical unit 59 may further include a second rotating unit (not illustrated) that rotates with the center of a light irradiating unit (not illustrated) as a rotation axis while being coupled to the side surface of the internal space. The second rotating unit may rotate the light irradiating unit and first and second cameras in a third rotation direction M3.

The three-dimensional scanners 10 and 50 may transmit the obtained raw data to the intraoral image processing apparatus 100 through the communication network 30. The raw data obtained by the three-dimensional scanners 10 and 50 may be transmitted to the intraoral image processing apparatus 100 connected through the wired or wireless communication network 30. In an embodiment, the three-dimensional scanners 10 and 50 may transmit the two-dimensional intraoral image to the intraoral image processing apparatus 100 through the communication network 30.

The intraoral image processing apparatus 100 may be connected to the three-dimensional scanners 10 and 50 through the wired or wireless communication network 30 and may receive, from the three-dimensional scanners 10 and 50, the two-dimensional intraoral image or the raw data obtained by scanning the object. The intraoral image processing apparatus 100 may be any electronic device that may generate, process, display, and/or transmit a three-dimensional intraoral image based on the received raw data. Also, the intraoral image processing apparatus 100 may be any electronic device that may generate, process, display, and/or transmit a three-dimensional intraoral image based on the received two-dimensional intraoral image.

The intraoral image processing apparatus 100 may be, but is not limited to, a computing device such as a smart phone, a laptop computer, a desktop computer, a PDA, or a tablet PC. Also, the intraoral image processing apparatus 100 may be provided in the form of a server (or a server device) for processing a three-dimensional intraoral image.

The intraoral image processing apparatus 100 may generate a three-dimensional intraoral image or generate information by processing the raw data or the two-dimensional intraoral image received from the three-dimensional scanners 10 and 50. The intraoral image processing apparatus 100 may display the generated information and three-dimensional intraoral image through a display 130.

When the three-dimensional scanners 10 and 50 transmits the raw data obtained through scanning to the intraoral image processing apparatus 100, the intraoral image processing apparatus 100 may generate a three-dimensional intraoral image three-dimensionally representing the oral cavity based on the received raw data.

When the three-dimensional scanners 10 and 50 transmits the two-dimensional intraoral image obtained through scanning to the intraoral image processing apparatus 100, the intraoral image processing apparatus 100 may generate a three-dimensional intraoral image three-dimensionally representing the oral cavity based on the received two-dimensional intraoral image.

In an embodiment, based on the received raw data or two-dimensional intraoral image, the intraoral image processing apparatus 100 may generate three-dimensional data (e.g., surface data or mesh data) that three-dimensionally represents the shape of the surface of the object.

Also, a ‘three-dimensional image’ may be generated by three-dimensionally modeling the object based on the received raw data or two-dimensional intraoral image and therefore may be referred to as a ‘three-dimensional model’. Hereinafter, a model or an image two-dimensionally or three-dimensionally representing the object will be collectively referred to as an ‘intraoral image’.

Also, the intraoral image processing apparatus 100 may analyze, process, display, and/or transmit the generated intraoral image to an external device. In an embodiment, the intraoral image processing apparatus 100 may be an electronic device that may generate and display an intraoral image three-dimensionally representing the object.

In an embodiment, tooth preparation may be performed to generate a prosthesis for dental treatment.

The tooth preparation may refer to a process of generating a space for a planned restoration material by cutting a tooth by removing a structurally unsound portion or removing a tooth decay in order to restore a restoration target tooth to its original form and function and may also be briefly referred to as “preparation”.

In an embodiment, a tooth before tooth preparation may be referred to as a “pre-preparation tooth”. A tooth that has undergone tooth preparation may be referred to as a “prepared tooth”.

In an embodiment, a dental prosthesis may refer to a prosthesis that may artificially replace a tooth when the tooth is lost; for example, a crown may refer to a tooth cap that is a type of dental restoration that completely covers or surrounds a tooth or implant.

In an embodiment, when the intraoral scanner 10 scans a prepared tooth, the intraoral image processing apparatus 100 may obtain an intraoral image including the prepared tooth. When the intraoral scanner 10 scans a pre-preparation tooth, the intraoral image processing apparatus 100 may obtain an intraoral image including the pre-preparation tooth.

In an embodiment, the intraoral image processing apparatus 100 according to the present disclosure may obtain an intraoral image including a tooth. In an embodiment, the intraoral image may include a prepared tooth. Hereinafter, for convenience of description, the intraoral image will be described as including a prepared tooth.

In an embodiment, when an intraoral image including a prepared tooth is obtained, the intraoral image processing apparatus 100 may set an insertion direction of a prosthesis corresponding to the prepared tooth and obtain an undercut area included in the prepared tooth based on the set insertion direction. In order to facilitate insertion of the prosthesis, the intraoral image processing apparatus 100 may compensate the undercut area included in the prepared tooth based on the set insertion direction.

However, the present disclosure is not limited thereto. The intraoral image processing apparatus 100 may set an insertion direction of a prosthesis corresponding to an unprepared tooth and obtain an undercut area included in the unprepared tooth based on the set insertion direction. In order to facilitate insertion of the prosthesis, the intraoral image processing apparatus 100 may compensate the undercut area included in the unprepared tooth based on the set insertion direction.

That is, the intraoral image processing apparatus 100 and an intraoral image processing method according to the present disclosure may operate based on an intraoral image including a tooth, regardless of a prepared tooth or an unprepared tooth. Hereinafter, for convenience of description, only the operation of the intraoral image processing apparatus 100 and the intraoral image processing method based on an intraoral image including a prepared tooth will be described.

In an embodiment, when the intraoral image processing apparatus 100 according to the present disclosure obtains an intraoral image including a pre-preparation tooth, the intraoral image processing apparatus 100 may generate a prosthesis and an inner shape of the prosthesis by applying a preset reference insertion direction to the intraoral image. In this case, a prosthesis target tooth may be referred to as an unprepared tooth; however, the present disclosure is not limited thereto. The intraoral image processing apparatus 100 may generate a prosthesis having an outer surface (eggshell) of the prosthesis target tooth and an inner shape of the prosthesis by applying a preset reference insertion direction to the prosthesis target tooth. In the present disclosure, the preset reference insertion direction may be, for example, a normal direction of the prosthesis target tooth.

The intraoral image processing apparatus 100 may set an insertion direction of the prosthesis based on the shape of the prosthesis target tooth and change an inner shape of the prosthesis based on the set insertion direction.

In this case, the prosthesis target tooth may be an abutment that may support the prosthesis when the prosthesis is inserted thereinto. In an embodiment, the abutment may be formed by preparing a pre-preparation tooth. When the intraoral image processing apparatus 100 obtains an intraoral image including a prepared tooth, it may be an image obtained by scanning an already prepared tooth by using the intraoral scanner 10.

When the intraoral image processing apparatus 100 obtains an intraoral image including a pre-preparation tooth, it may be an image obtained by scanning an unprepared tooth by using the intraoral scanner 10. The intraoral image processing apparatus 100 may generate a prosthesis of the prosthesis target tooth by applying a reference insertion direction to an image of the unprepared tooth.

The intraoral image processing apparatus 100 and the operation thereof according to the present disclosure will be described in detail with reference to the accompanying drawings.

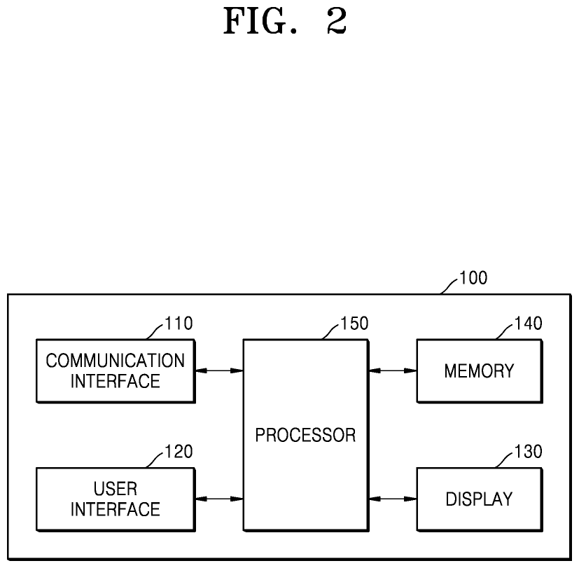

FIG. 2 is a block diagram for describing an intraoral image processing system according to an embodiment of the present disclosure.

Referring to FIG. 2, the intraoral image processing apparatus 100 may include a communication interface 110, a user interface 120, a display 130, a memory 140, and at least one processor 150.

The communication interface 110 may communicate with at least one external electronic device (e.g., the intraoral scanner 10 (see FIG. 1), the table scanner 50 (see FIG. 1), a server, or an external medical device) through a wired or wireless communication network. The communication interface 110 may communicate with at least one external electronic device under the control by the at least one processor 150.

Particularly, the communication interface 110 may include at least one short-range communication module communication according to the communication standard such as Bluetooth, WiFi, Bluetooth Low Energy (BLE), NFC/RFID, WiFi Direct, UWB, or ZigBee.

Also, the communication interface 110 may further include a long-range communication module for performing communication with a server to support long-range communication according to the long-range communication standard. Particularly, the communication interface 110 may include a long-range communication module performing communication through a network for Internet communication. Also, the communication interface 110 may include a long-range communication module performing communication through a communication network conforming to the communication standard such as 3G, 4G, and/or 5G.

Also, in order to communicate by wire with an external electronic device (e.g., an intraoral scanner), the communication interface 110 may include at least one port for being connected to the external electronic device through a wired cable. Accordingly, the communication interface 110 may communicate with the external electronic device connected by wire through the at least one port.

The user interface 120 may receive a user input for controlling the intraoral image processing apparatus 100. The user interface 120 may include, but is not limited to, a user input device including a touch panel for sensing a user's touch, a button for receiving a user's push operation, and/or a mouse or a keyboard for designating or selecting a point on a user interface screen.

Also, the user interface 120 may include a voice recognition device for voice recognition. For example, the voice recognition device may be a microphone, and the voice recognition device may receive a user's voice command or voice request. Accordingly, the at least one processor 150 may control an operation corresponding to the voice command or voice request to be performed.

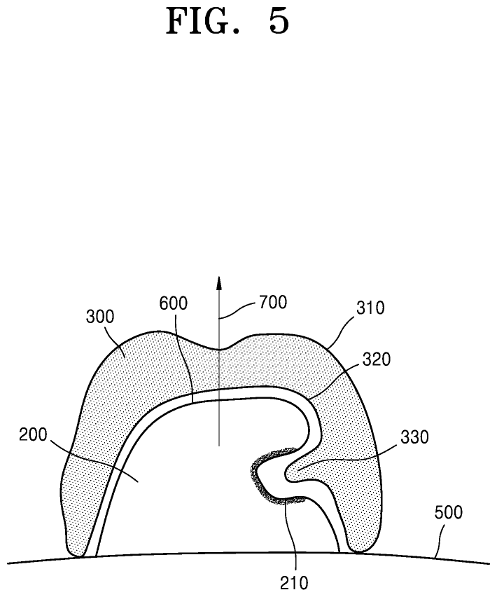

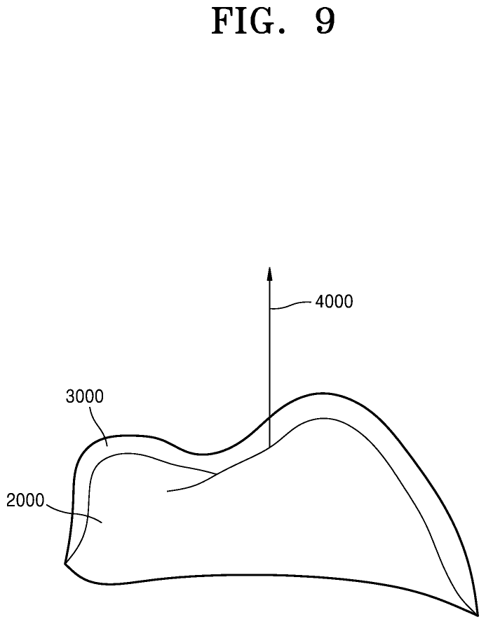

The user interface 120 according to an embodiment may receive a user input for compensating an undercut area 210 included in a prepared tooth 200 (see FIG. 5) described below. Also, the user interface 120 may receive a user input for changing an inner shape 2000 (see FIG. 9) of a prosthesis corresponding to a preset reference insertion direction 4000 (see FIG. 9) described below.

The display 130 may display a screen. Particularly, the display 130 may display a certain screen under the control by the at least one processor 150. Particularly, the display 130 may display a user interface screen including a three-dimensional intraoral image generated based on the data obtained by scanning the patient's oral cavity by using the intraoral scanner 10. Alternatively, the display 130 may display a user interface screen including a three-dimensional intraoral image of the object generated based on the data obtained from the table scanner 50.

Alternatively, the display 130 may display a user interface screen including information related to the patient's dental treatment.

In an embodiment, the memory 140 may store at least one instruction executed by the at least one processor 150. The memory 140 may store at least one program executed by the at least one processor 150. In an embodiment, the memory 140 may store the data received from the three-dimensional scanners 10 and 50 (e.g., the raw data and two-dimensional intraoral image obtained through scanning). The memory 140 may store the three-dimensional intraoral image three-dimensionally representing the object.

The at least one processor 150 may execute at least one instruction stored in the memory 140 to perform control such that a desired operation may be performed. Here, the at least one instruction may be stored in an internal memory included in the at least one processor 150.

Particularly, by executing at least one instruction stored in the memory 140, the at least one processor 150 may control at least one component included in the intraoral image processing apparatus 100 such that a desired operation may be performed. Thus, describing a case where the at least one processor 150 performs certain operations as an example may mean that the at least one processor 150 controls at least one component included in the intraoral image processing apparatus 100 such that certain operations may be performed.

In an embodiment, by executing the at least one instruction included in the memory 140, the at least one processor 150 may obtain an intraoral image including a prepared tooth. In an embodiment, by executing the at least one instruction included in the memory 140, the at least one processor 150 may set an insertion direction of a prosthesis corresponding to the prepared tooth. In an embodiment, by executing the at least one instruction included in the memory 140, the at least one processor 150 may obtain an undercut area included in the prepared tooth based on the set insertion direction and the intraoral image. In an embodiment, by executing the at least one instruction included in the memory 140, the at least one processor 150 may compensate the undercut area based on the set insertion direction.

In an embodiment, by executing the at least one instruction included in the memory 140, the at least one processor 150 may set the insertion direction based on the shape of the prepared tooth. In this case, when referring to the accompanying drawings and the description of the present disclosure, for convenience of description, the insertion direction will be described as meaning the direction from the bottom surface of the prepared tooth toward the occlusal surface of the prepared tooth. However, the present disclosure is not limited thereto. The insertion direction may also mean the direction from the occlusal surface of the prepared tooth toward the bottom surface of the prepared tooth. The intraoral image processing apparatus 100 and the intraoral image processing method of the present disclosure may operate even when the insertion direction means the direction from the occlusal surface of the prepared tooth toward the bottom surface of the prepared tooth.

In an embodiment, the prepared tooth may include a plurality of points along the surface shape of the prepared tooth. By executing the at least one instruction included in the memory 140, in obtaining the undercut area, the at least one processor 150 may provide a virtual line in a direction parallel to the insertion direction from each of the plurality of points. By executing at least one command included in the memory 140, the at least one processor 150 may obtain, as the undercut area, an area of the prepared tooth including at least one point providing a virtual line intersecting the prepared tooth.

In an embodiment, the at least one point included in the undercut area may be defined as a first reference point, at least one point meeting the virtual line provided from the first reference point may be defined as a second reference point, and a center of an extension line passing through the first reference point and the second reference point may be defined as a third reference point. By executing the at least one instruction included in the memory 140, in compensating the undercut area, the at least one processor 150 may obtain a center point located at the center of the prepared tooth and obtain a reference direction from the center point toward the third reference point. By executing the at least one instruction included in the memory 140, the at least one processor 150 may move the first reference point in the same direction as the reference direction to compensate the undercut area.

In an embodiment, by executing the at least one instruction included in the memory 140, before moving the first reference point, the at least one processor 150 may move the center point to be on the same line as the first reference point.

In an embodiment, by executing the at least one instruction included in the memory 140, in compensating the undercut area, the at least one processor 150 may calculate a normal direction toward the first reference point and the outside of the prepared tooth and move the center point to be on the same line as the first reference point.

In an embodiment, by executing the at least one instruction included in the memory 140, in compensating the undercut area, the at least one processor 150 may calculate a normal direction from the first reference point toward the outside of the prepared tooth. When an angle between a vector with the reference direction and a vector with the normal direction is greater than 90 degrees, the at least one processor 150 may compensate the undercut area by moving the first reference point in a direction opposite to the reference direction.

In an embodiment, by executing the at least one instruction included in the memory 140, the at least one processor 150 may compensate the undercut area by moving at least one point included in the undercut area in a direction perpendicular to the insertion direction and toward the outside of the prepared tooth.

In an embodiment, by executing the at least one instruction included in the memory 140, the at least one processor 150 may repeat an operation of compensating the undercut area until a virtual line provided from each of the plurality of points included in the prepared tooth does not intersect the prepared tooth.

In an embodiment, by executing the at least one instruction included in the memory 140, after compensating the undercut area, the at least one processor 150 may further perform an operation of simplifying and smoothing the compensated undercut area.

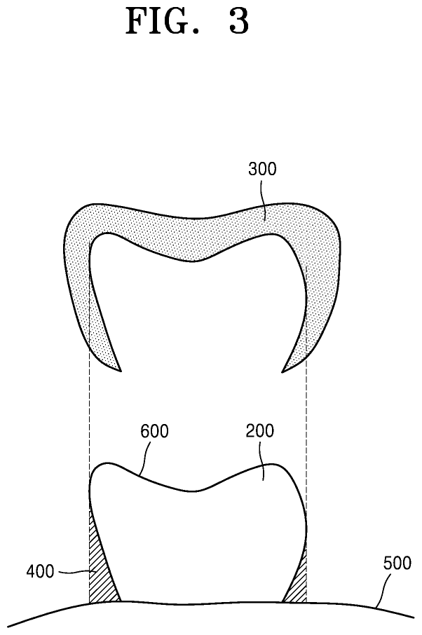

An operation of obtaining the undercut area included in the prepared tooth and compensating the undercut area based on the set insertion direction will be described below with reference to FIGS. 3 to 7.

In an embodiment, by executing the at least one instruction included in the memory 140, the at least one processor 150 may obtain an intraoral image including a prosthesis target tooth. In an embodiment, by executing the at least one instruction included in the memory 140, the at least one processor 150 may generate a prosthesis and an inner shape of the prosthesis by applying a preset reference insertion direction to the intraoral image. In an embodiment, by executing the at least one instruction included in the memory 140, the at least one processor 150 may set an insertion direction of the prosthesis based on the shape of the prosthesis target tooth. In an embodiment, by executing the at least one instruction included in the memory 140, the at least one processor 150 may compensate an undercut area according to the insertion direction by changing the inner shape of the prosthesis based on the set insertion direction.

In an embodiment, by executing the at least one instruction included in the memory 140, in changing the inner shape of the prosthesis, the at least one processor 150 may generate a virtual inner shape by applying the set insertion direction to the intraoral image and change the inner shape of the prosthesis by comparing the inner shape of the prosthesis with the virtual inner shape. In an embodiment, a margin line of the inner shape of the prosthesis and a margin line of the virtual inner shape may be the same as each other. The inner shape of the prosthesis may have a shape extending from the margin line in the reference insertion direction, and the virtual inner shape may have a shape extending from the margin line in the set insertion direction.

In an embodiment, the inner shape of the prosthesis may be the shape of the prepared tooth.

In an embodiment, the inner shape of the prosthesis may include a plurality of points along the surface of the inner shape. In an embodiment, by executing the at least one instruction included in the memory 140, in changing the inner shape of the prosthesis, the at least one processor 150 may provide a virtual line in a normal direction from each of the plurality of points toward the inside of the inner shape of the prosthesis and change a shape of an area in the inner shape of the prosthesis, which includes at least one point providing a virtual line intersecting the virtual inner shape, to correspond to the virtual inner shape.

In an embodiment, by executing the at least one instruction included in the memory 140, the at least one processor 150 may change the inner shape of the prosthesis and simplify and smooth the changed inner shape of the prosthesis.

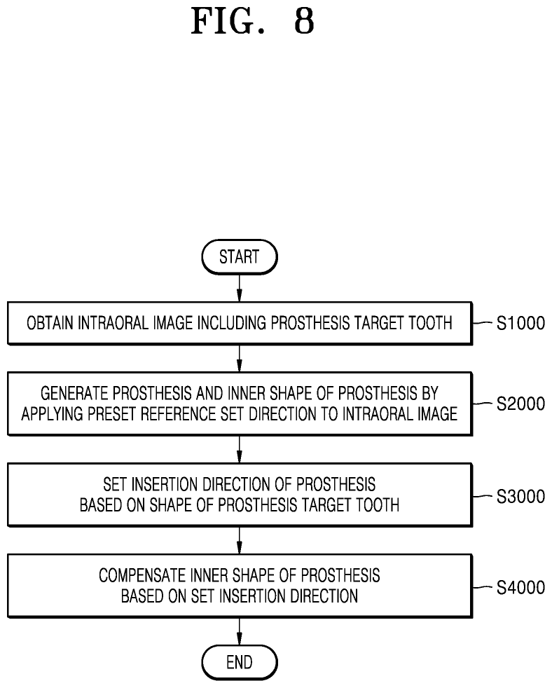

An operation of generating the prosthesis and the inner shape of the prosthesis by applying the preset reference insertion direction to the image of the prosthesis target tooth, setting the insertion direction of the prosthesis based on the shape of the prosthesis target tooth, and changing the inner surface of the prosthesis based on the set insertion direction will be described below with reference to FIGS. 8 to 10C.

The at least one processor 150 according to an embodiment may internally include at least one internal processor and a memory device (e.g., RAM or ROM) for storing at least one of programs, instructions, signals, and data to be processed or used by the internal processor.

Also, the at least one processor 150 may include a graphic processor (graphic processing unit) for graphic processing corresponding to video. Also, the at least one processor 150 may be implemented as a System-on-Chip (SoC) including a combination of a core and a GPU. Also, the at least one processor 150 may include a single core or a multi core. For example, the at least one processor 150 may include a dual core, a triple core, a quad core, a hexa core, an octa core, a deca core, a dodeca core, a hexadecimal core, and/or the like.

In an embodiment, the at least one processor 150 may generate a three-dimensional image based on the raw data or two-dimensional image received from the three-dimensional scanners 10 and 50.

Particularly, under the control by the at least one processor 150, the communication interface 110 may receive the raw data or two-dimensional image obtained by the three-dimensional scanners 10 and 50. Based on the raw data and two-dimensional image received by the communication interface 110, the at least one processor 150 may generate a three-dimensional image three-dimensionally representing the object. For example, in order to restore a three-dimensional image according to an optical triangulation method, the three-dimensional scanners 10 and 50 may include an L camera corresponding to the left field of view and an R camera corresponding to the right field of view. The three-dimensional scanners 10 and 50 may obtain L image data corresponding to the left field of view and R image data corresponding to the right field of view from the L camera and the R camera, respectively. The three-dimensional scanners 10 and 50 may transmit raw data including the L image data and the R image data to the communication interface 110 of the intraoral image processing apparatus 100. In an embodiment, the three-dimensional scanners 10 and 50 may generate a two-dimensional image based on the raw data and transmit the generated two-dimensional image to the communication interface 110 of the intraoral image processing apparatus 100.

The communication interface 110 may transmit the received raw data or two-dimensional image to the at least one processor 150, and the at least one processor 150 may generate a three-dimensional image based on the received raw data or two-dimensional image.

Also, the at least one processor 150 may control the communication interface 110 to directly receive a three-dimensional image three-dimensionally representing the object from an external server, a medical device, or the like. In this case, the at least one processor 150 may obtain a three-dimensional image from the outside instead of generating a three-dimensional image based on the raw data.

According to embodiments, the at least one processor 150 performing operations such as “extracting”, “obtaining”, and “generating” may include not only the at least one processor 150 executing at least one instruction to directly perform the above operations but also the at least one processor 150 controlling other components to perform the above operations.

In order to perform an operation of the intraoral image processing apparatus 100 according to the present disclosure, the intraoral image processing apparatus 100 may include only some of the components illustrated in FIG. 2 or may include more components in addition to the components illustrated in FIG. 2.

Also, the intraoral image processing apparatus 100 may store and execute dedicated software linked to the three-dimensional scanners 10 and 50. Here, the dedicated software may also be referred to as a dedicated program, a dedicated tool, or a dedicated application. When the intraoral image processing apparatus 100 operates in conjunction with the three-dimensional scanners 10 and 50, the dedicated software stored in the intraoral image processing apparatus 100 may be connected to the three-dimensional scanners 10 and 50 to receive in real time the data obtained by scanning the object. In an embodiment, there is dedicated software for processing the data obtained by scanning the object by using an intraoral scanner of Medit. Particularly, Medit produces and distributes dedicated software (e.g., Medit Link) as software for processing, managing, using, and/or transmitting the data obtained by the three-dimensional scanner (e.g., i500). Here, because the “dedicated software” refers to a program, a tool, or an application operable in conjunction with the three-dimensional scanner, various three-dimensional scanners developed and sold by various manufacturers may be used in common. Also, the above dedicated software may be produced and distributed separately from the three-dimensional scanner for scanning the object.

The intraoral image processing apparatus 100 may store and execute dedicated software corresponding to the three-dimensional scanner. The dedicated software may perform one or more operations for obtaining, processing, storing, and/or transmitting an image. Here, the dedicated software may be stored in the processor. Also, the dedicated software may provide a user interface for using the data obtained by the three-dimensional scanner. Here, the user interface screen provided by the dedicated software may include an image generated according to the embodiments.

FIG. 3 is a diagram for describing an undercut area according to an embodiment of the present disclosure.

Referring to FIG. 3, a prepared tooth 200 and a prosthesis 300 corresponding to the prepared tooth 200 are illustrated in FIG. 3. In an embodiment, the prepared tooth 200 may include a bottom surface contacting a gingiva 500 and an occlusal surface 600. The prosthesis 300 may be coupled to the prepared tooth 200 toward the occlusal surface of the prepared tooth 200 to cover or surround the prepared tooth 200.

In an embodiment, in the prepared teeth 200, an area corresponding to the space between a height of contour of the prepared tooth 200 and the gingiva 500 may be defined as an undercut area 400. In an embodiment, when the prepared tooth 200 includes the undercut area 400, the prosthesis 300 may have a shape for covering or surrounding the undercut area 400. In FIG. 3, the prosthesis 300 is illustrated as including a shape for covering or surrounding the undercut area 400.

In this case, because the prosthesis 300 is inserted into the undercut area 400 through the height of the contour of the prepared tooth 200, it may be difficult to couple the prosthesis 300 to the prepared tooth 200 and the patient may feel uncomfortable after the prosthesis 300 is inserted thereinto. Thus, when the prepared tooth 200 includes the undercut area 400, it may be necessary to facilitate coupling the prosthesis 300 to the prepared tooth 200 through an operation of compensating the undercut area 400.

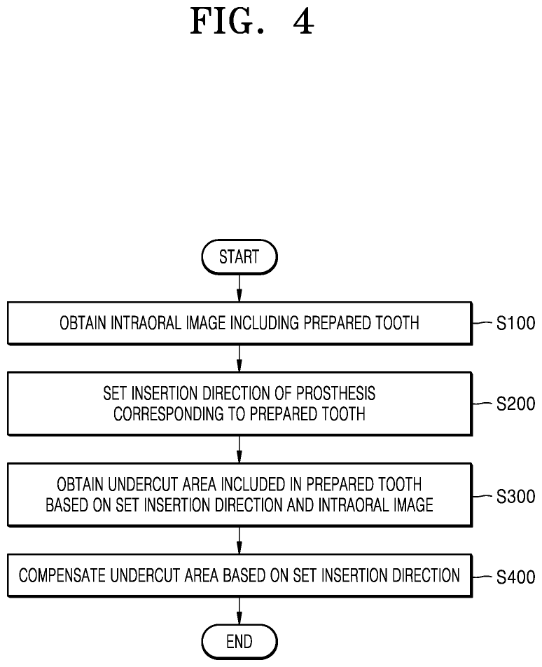

FIG. 4 is a flowchart for describing an intraoral image processing method according to an embodiment of the present disclosure.

Referring to FIGS. 1 and 4, in an embodiment, an operating method of the intraoral image processing apparatus 100 may include an operation S100 of obtaining an intraoral image including a prepared tooth. The operating method of the intraoral image processing apparatus 100 may include an operation S200 of setting an insertion direction of a prosthesis corresponding to the prepared tooth. In an embodiment, the operation S200 of setting the insertion direction of the prosthesis may include an operation of setting the insertion direction based on the shape of the prepared tooth. In this case, the insertion direction may be a direction from the bottom surface of the prepared tooth toward the occlusal surface of the prepared tooth. The insertion direction may refer to an average normal direction of the prepared tooth.

The operation S200 of setting the insertion direction of the prosthesis will be described below with reference to FIG. 5.

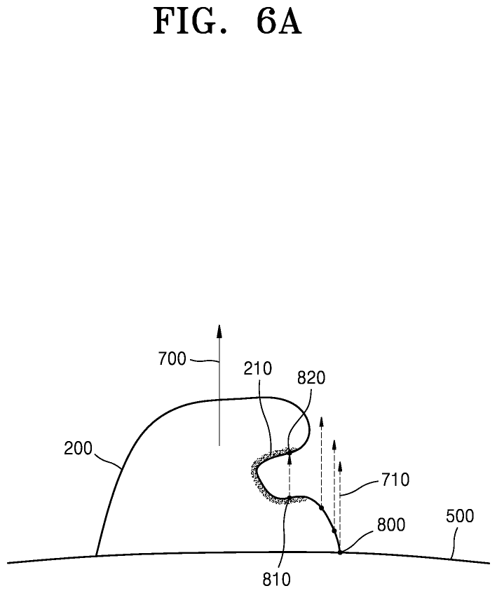

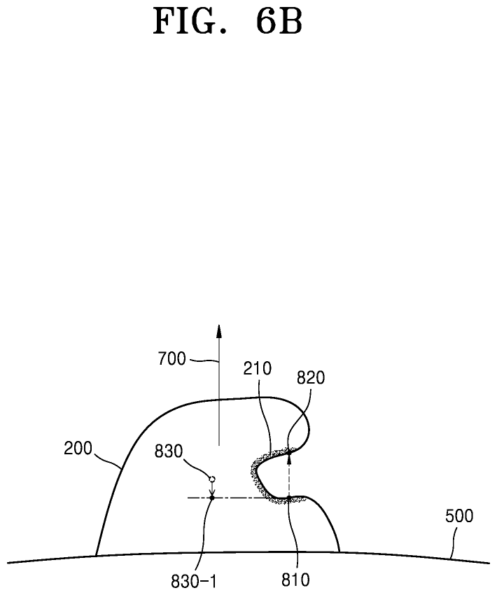

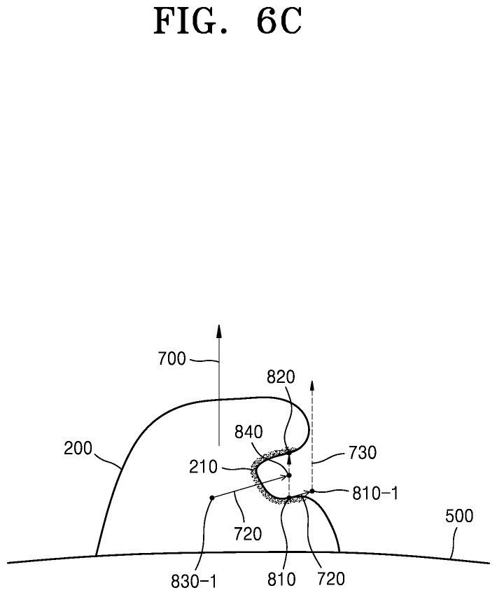

In an embodiment, the operating method of the intraoral image processing apparatus 100 may include an operation S300 of obtaining an undercut area included in the prepared tooth based on the set insertion direction and the intraoral image. In an embodiment, the prepared tooth may include a plurality of points along the surface shape of the prepared tooth. The operation S300 of obtaining the undercut area may include an operation of providing a virtual line in a direction parallel to the insertion direction from each of the plurality of points and an operation of obtaining, as the undercut area, an area of the prepared tooth including at least one point providing a virtual line intersecting the prepared tooth. The operation S300 of obtaining the undercut area will be described below with reference to FIG. 6A.

In an embodiment, the operating method of the intraoral image processing apparatus 100 may include an operation S400 of compensating the undercut area based on the set insertion direction. In an embodiment, the operation S400 of compensating the undercut area may include an operation of obtaining a center point located at the center of the prepared tooth, an operation of obtaining a reference direction from the center point toward the third reference point, and an operation of moving the first reference point in the same direction as the reference direction to compensate the undercut area. In an embodiment, the operation S400 of compensating the undercut area may further include, before the operation of moving the first reference point, an operation of moving the center point to be on the same line as the first reference point. The operation of obtaining the center point, moving the center point to be on the same line as the first reference point, obtaining the reference direction toward the center point and the third reference point, and moving the first reference point in the reference direction will be described below in FIGS. 6B and 6C.

In an embodiment, the operation S400 of compensating the undercut area may further include an operation of calculating a normal direction from the first reference point toward the outside of the prepared tooth and an operation of compensating the undercut area by moving the first reference point in a direction opposite to the reference direction when an angle between a vector with the reference direction and a vector with the normal direction is greater than 90 degrees.

In an embodiment, the operation S400 of compensating the undercut area may further include an operation of moving at least one point included in the undercut area in a direction perpendicular to the insertion direction and toward the outside of the prepared tooth, to compensate the undercut area.

In an embodiment, in the operating method of the intraoral image processing apparatus 100, the operation S400 of compensating the undercut area may be repeated until a virtual line provided from each of the plurality of points does not intersect the prepared tooth in the operation S300 of obtaining the undercut area.

In an embodiment, the operating method of the intraoral image processing apparatus 100 may further include, after the operation S400 of compensating the undercut area, simplifying and smoothing the compensated undercut area.

FIG. 5 is a diagram for describing an undercut area and a prosthesis according to an embodiment of the present disclosure. Hereinafter, the same reference numerals will be assigned to the same elements as those described in FIG. 3, and redundant descriptions thereof will be omitted for conciseness.

Referring to FIG. 5, the prosthesis 300 may be represented as including a prosthesis outer surface 310 and a prosthesis inner surface 320. In an embodiment, based on an insertion direction 700, the prosthesis 300 may be coupled to the prepared tooth 200 such that the prosthesis inner surface 320 may face the occlusal surface 600 of the prepared tooth 200.

In an embodiment, the insertion direction 700 may be set according to the shape of the prepared tooth 200, the shape of adjacent teeth around the prepared tooth 200, or the arrangement between the prepared tooth 200 and the adjacent teeth around the prepared tooth 200. Also, the insertion direction 700 may represent an average normal direction of the prepared tooth 200.

In an embodiment, the prepared tooth 200 may include an undercut area 210 formed inside the prepared tooth 200. In FIG. 5, the undercut area 210 may be formed at the right side surface of the prepared tooth 200; however, the present disclosure is not limited thereto. The undercut area 210 may be formed at the left side surface or upper surface of the prepared tooth 200. Also, the prepared tooth 200 may include two or more undercut areas. In an embodiment, the undercut area 210 illustrated in FIG. 5 may be exaggerated for convenience of description. The size of the undercut area 210 included in the prepared tooth 200 may be smaller than the size of the undercut area 210 illustrated in FIG. 5. Also, in FIG. 5, for convenience of description, the undercut area 210 is illustrated as being formed at a middle portion of the prepared tooth 200 spaced apart from a margin line of the prepared tooth 200; however, the present disclosure is not limited thereto. In an embodiment, the undercut area 210 included in the prepared tooth 200 may be formed at a lower end portion of the prepared tooth 200 adjacent to a margin line of the prepared tooth 200.

Also, in FIG. 5, the width of the prepared tooth 200 is illustrated as increasing toward the lower side of the prepared tooth 200; however, the present disclosure is not limited thereto. In an embodiment, as illustrated in FIG. 3, the width of the prepared tooth 200 may increase toward the upper side of the prepared tooth 200.

In an embodiment, when the prepared tooth 200 includes the undercut area 210, the prosthesis 300 may be generated to include a protrusion area 330 corresponding to the undercut area 210. In this case, when the prosthesis 300 is coupled to the prepared tooth 200 in the insertion direction 700, the protrusion area 330 may be caught on the upper surface of the prepared tooth 200 and thus it may be difficult to cover or surround the prepared tooth 200 with the prosthesis 300.

FIG. 6A is a diagram for describing an operation of compensating an undercut area according to an embodiment of the present disclosure. Hereinafter, for convenience of description, the same reference numerals will be assigned to the same elements as those described in FIGS. 3 and 5, and redundant descriptions thereof will be omitted for conciseness.

Referring to FIG. 6A, the prepared tooth 200 may include a plurality of points 800, 810, and 820 along the surface shape of the prepared tooth 200. In an embodiment, the at least one processor 150 (see FIG. 2) may provide a virtual line in a direction 710 parallel to the insertion direction 700 from each of the plurality of points 800, 810, and 820. The at least one processor 150 may obtain, among the plurality of points 800, 810, and 820, at least one point 810 at which a virtual line provided from each point intersects the prepared tooth 200.

In an embodiment, the plurality of points 800, 810, and 820 may include a first point 800, a second point 810, and a third point 820. A virtual line provided in the direction 710 parallel to the insertion direction 700 from the first point 800 may not intersect the prepared tooth 200. A virtual line provided in the direction 710 parallel to the insertion direction 700 from the second point 810 may intersect the third point 820 of the prepared tooth 200. Although not illustrated in FIG. 6A, a virtual line provided in the direction 710 parallel to the insertion direction 700 from the third point 820 may also intersect the prepared tooth 200.

The at least one processor 150 may obtain, among the plurality of points 800, 810, and 820, the first point 810 and the third point 820 at which a virtual line provided from each point intersects the prepared tooth 200.

In an embodiment, when the prepared tooth 200 intersects a virtual line provided in a direction parallel to the insertion direction 700, an area between a point providing the virtual line and a point intersected by the virtual line may be an area having a narrower width than the height of contour of the prepared tooth 200. Thus, the at least one processor 150 may obtain, as the undercut area 210, an area of the prepared tooth 200 including at least one point providing a virtual line intersecting the prepared tooth 200.

In an embodiment, the at least one processor 150 may obtain, as the undercut area 210, an area including the second and third points 810 and 820 among the first to third points 800, 810, and 820. However, the present disclosure is not limited thereto, and the undercut area 210 may include three or more points.

Hereinafter, for convenience of description, the second point 810 will be referred to as a first reference point 810, and the third point 820 will be referred to as a second reference point 820.

FIG. 6B is a diagram for describing an operation of compensating an undercut area according to an embodiment of the present disclosure. Hereinafter, for convenience of description, the same reference numerals will be assigned to the same elements as those described in FIG. 6A, and redundant descriptions thereof will be omitted for conciseness.

Referring to FIG. 6B, the prepared tooth 200 may include a center point 830 located at the center of the prepared tooth 200. In an embodiment, the center of the prepared tooth 200 may be obtained based on a mesh constituting the prepared tooth 200. A bounding box in the shape of a rectangular parallelepiped may be formed by connecting a point having the maximum coordinate values of the mesh constituting the prepared tooth 200 to a point having the minimum coordinate values thereof, and a diagonal center of the bounding box may be defined as the center of the prepared tooth 200. In an embodiment, when three coordinate axes of the three-dimensional space in which the prepared tooth 200 is arranged are an X axis, a Y axis, and a Z axis that are perpendicular to each other, the point having the maximum coordinate values of the mesh may refer to a point of the prepared tooth 200 having the maximum coordinate value in each of the X axis, the Y axis, and the Z axis. In an embodiment, the point having the minimum coordinate values of the mesh may refer to a point of the prepared teeth 200 having the minimum coordinate value in each of the X axis, the Y axis, and the Z axis. In an embodiment, the bounding box may refer to a box that includes all of the meshes constituting the prepared tooth 200 and has the smallest size.

In an embodiment, the bounding box may be formed by using at least one of an Axis-Aligned Bounding Box algorithm, an Oriented Bounding Box algorithm, a Convex Hull algorithm, a Bounding Sphere algorithm, or a K-Discrete Oriented Polytope (K-DOP) algorithm. In an embodiment, the bounding box may be formed by using an 8-DOP algorithm forming a polyhedron with eight faces, among the K-DOP algorithms.

In an embodiment, in order to compensate the undercut area 210, before moving the first reference point 810, the at least one processor 150 may move the center point 830 to be on the same line as the first reference point 810. In an embodiment, a moved center point 830-1 may be located on the same line as the first reference point 810.

FIG. 6C is a diagram for describing an operation of compensating an undercut area according to an embodiment of the present disclosure. Hereinafter, for convenience of description, the same reference numerals will be assigned to the same elements as those described in FIGS. 6A and 6B, and redundant descriptions thereof will be omitted for conciseness.

Referring to FIG. 6C, the center of an extension line passing through the first reference point 810 and the second reference point 820 may be defined as a third reference point 840. In an embodiment, the direction toward the moved center point 830-1 and the third reference point 840 may be defined as a reference direction 720.

After obtaining the reference direction 720, the at least one processor 150 may move the first reference point 810 in a direction 720 parallel to the reference direction 720 to compensate the undercut area 210. In an embodiment, the unit by which the at least one processor 150 moves the first reference point 810 may be a preset unit mesh size. In an embodiment, the at least one processor 150 may compensate the undercut area 210 by repeatedly moving the first reference point 810 by the unit mesh size. In an embodiment, when the prepared tooth 200 includes a plurality of polygonal meshes that are repeated as triangular meshes, the unit mesh may be set to have a size corresponding to the average length of the edges included in the triangular meshes. In an embodiment, the unit mesh size may be set to 0.2 times the average length of the edges included in the triangular meshes constituting the prepared tooth 200.

In an embodiment, the at least one processor 150 may move the first reference point 810 in a direction 720 parallel to the reference direction 720 until a virtual line provided in a direction 730 parallel to the insertion direction 700 from the first reference point 810 does not meet the prepared tooth 200. Accordingly, the at least one processor 150 may compensate the undercut area 210.

In an embodiment, an area corresponding to a first reference point 810-1 moved in a direction 720 parallel to the reference direction 720 may be defined as a compensated undercut area. In an embodiment, the compensated undercut area may include not only a moved first reference point 810-1 but also at least one point moved from at least one point included in the undercut area 210.

In an embodiment, the at least one processor 150 may simplify and smooth the compensated undercut area. The compensated undercut area may include at least one point moved until a virtual line provided from at least one point included in the undercut area 210 does not intersect the prepared tooth 200. In this case, because the area of the undercut area 210 is greater than the area of the compensated undercut area, the distance between at least one moved point included in the compensated undercut area may be less than the distance between at least one point included in the undercut area 210. Thus, the at least one processor 150 may simplify and smooth the compensated undercut area to reduce the number of at least one moved point included in the compensated undercut area. Accordingly, the operation time required when the at least one processor 150 uses the compensated undercut area may be reduced.

However, the present disclosure is not limited thereto. The least one processor 150 may calculate a normal direction toward the outside of the prepared tooth 200 from at least one point included in the undercut area 210. When the angle between a vector with the reference direction 720 and a vector with the normal direction is greater than 90 degrees, the at least one processor 150 may obtain a compensated undercut area by moving at least one point included in the undercut area 210 in a direction opposite to the reference direction 720. In an embodiment, when the dot product of the vector with the reference direction 720 and the vector with the normal direction has a negative value, the at least one processor 150 may obtain a compensated undercut area by moving at least one point included in the undercut area 210 in a direction opposite to the reference direction 720.

In an embodiment, when the angle between the vector with the reference direction 720 and the vector with the normal direction is greater than 90 degrees, the undercut area 210 may be an area formed in a direction from the inside of the prepared tooth 200 toward the outside, that is, in the normal direction, in comparison with the prepared tooth 200 therearound. In an embodiment, when the dot product of the vector with the reference direction 720 and the vector with the normal direction has a negative value, the at least one processor 150 may obtain a compensated undercut area by compensating the undercut area 210 to be directed toward the inside of the prepared tooth 200, by moving at least one point included in the undercut area 210 in a direction opposite to the reference direction 720.

However, the present disclosure is not limited thereto. The at least one processor 150 may obtain a first reference direction from the center point 830 toward the first reference point 810 and obtain a compensated undercut area by moving at least one point included in the undercut area 210 in a direction opposite to the first reference direction when the angle between a vector with the first reference direction and a vector with the normal direction is greater than 90 degrees.

In an embodiment, the at least one processor 150 may not move the center point 830 to be on the same line as the first reference point 720. In this case, the at least one processor 150 may obtain a compensated undercut area by moving at least one point included in the undercut area 210 in a direction perpendicular to the insertion direction 700 and toward the outside of the prepared tooth 200. Without having to identify the center point 830 and the reference direction 720, after identifying the insertion direction 700 of the prosthesis 300 (see FIG. 5), the at least one processor 150 may move at least one point included in the undercut area 210 in a direction perpendicular to the insertion direction 700 and toward the outside of the prepared tooth 200. The at least one processor 150 may obtain a compensated undercut area by repeatedly moving the first reference point 720 by the unit mesh size.

However, the present disclosure is not limited thereto, and when the angle between a vector with the normal direction toward the outside of the prepared tooth 200 and a vector with the insertion direction 700 is greater than 90 degrees at the at least one point included in the undercut area 210, the at least one processor 150 may obtain a compensated undercut area by moving the at least one point included in the undercut area 210 in a direction perpendicular to the insertion direction 700 and toward the inside of the prepared tooth 200. In an embodiment, when the dot product of the vector with the normal direction toward the outside of the prepared tooth 200 and the vector with the insertion direction 700 has a negative value at the at least one point included in the undercut area 210, the at least one processor 150 may obtain a compensated undercut area by moving the at least one point included in the undercut area 210 in a direction perpendicular to the insertion direction 700 and toward the inside of the prepared tooth 200.

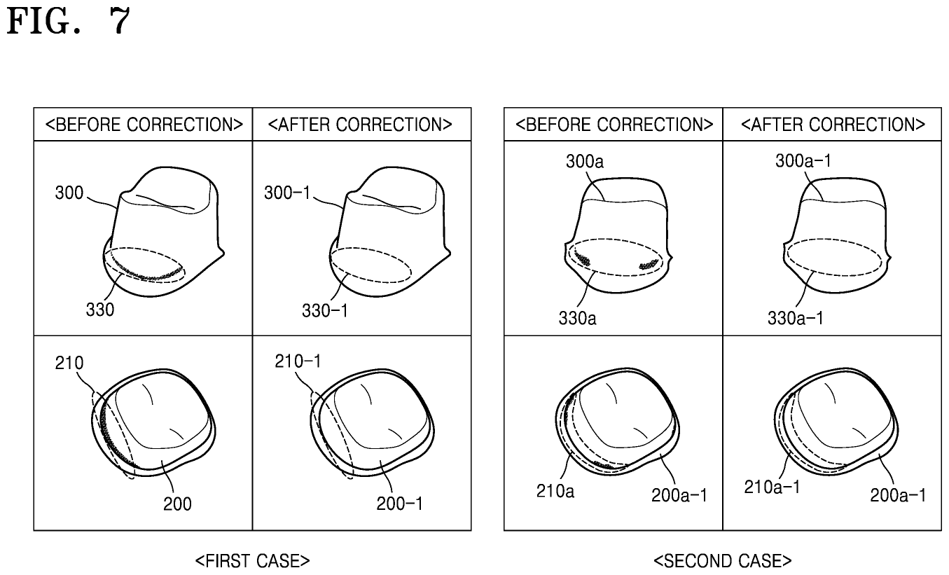

FIG. 7 is a diagram for describing an intraoral image processing apparatus and method according to an embodiment of the present disclosure.

Referring to FIG. 7, in <First Case>, an undercut area 210 included at the side surface of a pre-correction prepared tooth 200 and a corrected prepared tooth 200-1 including a corrected undercut area 210-1 are illustrated. According to the intraoral image processing apparatus 100 (see FIG. 1) and the operating method of the intraoral image processing apparatus 100 according to the present disclosure, the corrected undercut area 210-1 included in the corrected prepared tooth 200-1 is not directed toward the inside of the corrected prepared tooth 200-1 in comparison with the pre-correction undercut area 210.

Referring to FIG. 7, in <First Case>, a prosthesis 300 generated based on the pre-correction prepared tooth 200 and a correction prosthesis 300-1 generated based on the corrected prepared tooth 200-1 are illustrated. The prosthesis 300 includes a protrusion area 330 corresponding to the undercut area 210 of the pre-correction prepared tooth 200. The correction prosthesis 300-1 includes a correction protrusion area 330-1 corresponding to the corrected undercut area 210-1 included in the corrected prepared tooth 200-1. In an embodiment, the correction protrusion area 330-1 corresponding to the corrected undercut area 210-1 does not protrude toward the inside of the correction prosthesis 300-1. Thus, when the undercut area 210 is corrected according to the intraoral image processing apparatus 100 (see FIG. 1) and the operating method of the intraoral image processing apparatus 100 according to the present disclosure and the correction prosthesis 300-1 is generated corresponding to the corrected undercut area 210-1, the correction prosthesis 300-1 may easily cover or surround the corrected prepared tooth 200-1.

Referring to FIG. 7, in <Second Case>, an undercut area 210a included at a portion of the side surface of a pre-correction prepared tooth 200a and a corrected prepared tooth 200a including a corrected undercut area 210a-1 are illustrated. As in <First Case>, the corrected undercut area 210a-1 included in the corrected prepared tooth 200a-1 is not directed toward the inside of the corrected prepared tooth 200a-1 in comparison with the pre-correction undercut area 210a.

Referring to FIG. 7, in <Second Case>, a prosthesis 300a generated based on the pre-correction prepared tooth 200a and a correction prosthesis 300a-1 generated based on the corrected prepared tooth 200a-1 are illustrated. The prosthesis 300a includes a protrusion area 33a0 corresponding to the undercut area 210a of the pre-correction prepared tooth 200a. The correction prosthesis 300a-1 includes a correction protrusion area 330a-1 corresponding to the corrected undercut area 210a-1 included in the corrected prepared tooth 200a-1. As in <First Case>, the correction protrusion area 330a-1 corresponding to the corrected undercut area 210a-1 does not protrude toward the inside of the correction prosthesis 300a-1. Thus, the correction prosthesis 300a-1 may easily cover or surround the corrected prepared tooth 200a-1.

FIG. 8 is a flowchart for describing an intraoral image processing method according to an embodiment of the present disclosure.

Referring to FIGS. 1 and 8, in an embodiment, an operating method of the intraoral image processing apparatus 100 may include an operation S1000 of obtaining an intraoral image including a prosthesis target tooth. The operating method of the intraoral image processing apparatus 100 may include an operation S2000 of generating a shape of a prosthesis by applying a preset reference set direction to the intraoral image. In an embodiment, the reference set direction may refer to a preset insertion direction of the prosthesis. Hereinafter, for convenience of description, the reference set direction will be described as a reference insertion direction. The operating method of the intraoral image processing apparatus 100 may include an operation S3000 of setting an insertion direction of the prosthesis based on a shape of the prosthesis target tooth. The operating method of the intraoral image processing apparatus 100 may include an operation S4000 of changing an inner shape of the prosthesis based on the set insertion direction.

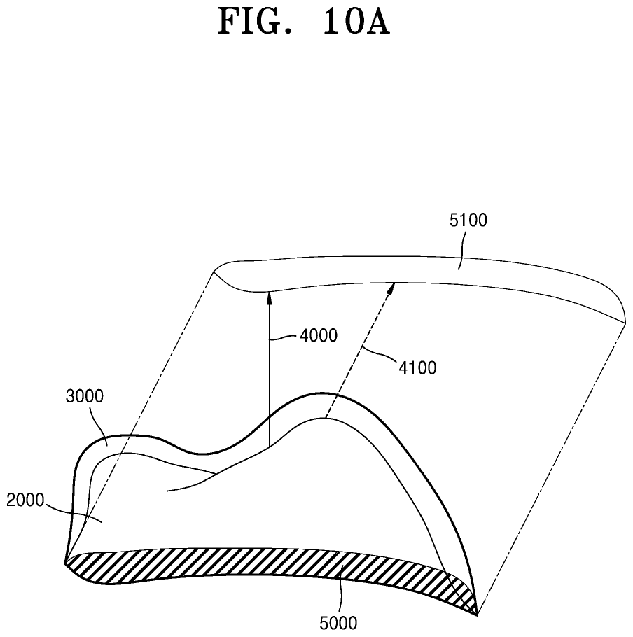

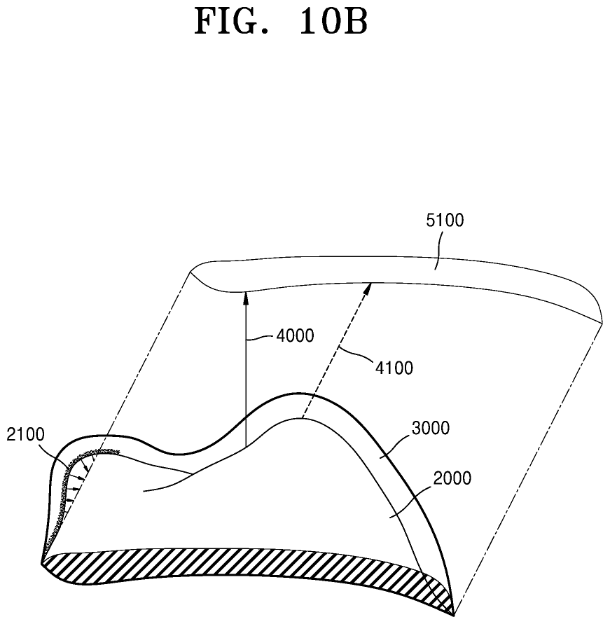

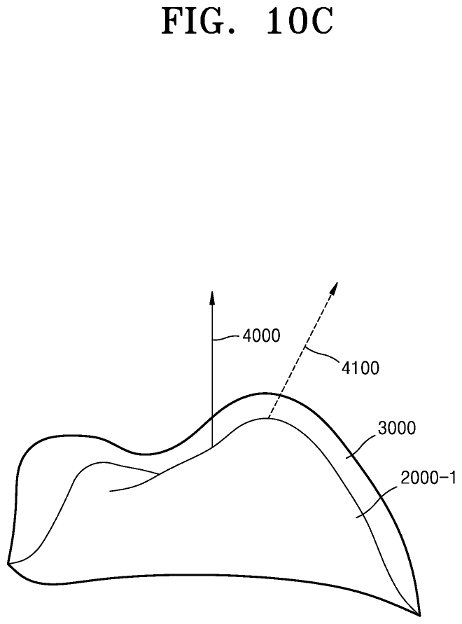

In an embodiment, the operation S4000 of changing the inner shape of the prosthesis may include an operation of generating a virtual inner shape by applying the set insertion direction to the intraoral image and an operation of changing the inner shape of the prosthesis by comparing the inner shape of the prosthesis with the virtual inner shape. In this case, a margin line of the inner shape of the prosthesis and a margin line of the virtual inner shape may be the same as each other. The inner shape of the prosthesis may have a shape extending from the margin line in the reference insertion direction, and the virtual inner shape may have a shape extending from the margin line in the set insertion direction. Hereinafter, the operation of generating the virtual inner shape will be described below with reference to FIG. 10A, and the operation of changing the inner shape of the prosthesis by comparing the inner shape of the prosthesis with the virtual inner shape will be described below with reference to FIG. 10B.

In an embodiment, the inner shape of the prosthesis may include a plurality of points along the surface of the inner shape. The operation S4000 of changing the inner shape of the prosthesis may include an operation of providing a virtual line in a normal direction toward an inside of the inner shape of the prosthesis from each of the plurality of points and an operation of changing a shape of an area in the inner shape of the prosthesis, which includes at least one point providing a virtual line intersecting the virtual inner shape, to correspond to the virtual inner shape. The operation S4000 of changing the inner shape of the prosthesis will be described below with reference to FIG. 10B.

In an embodiment, the operating method of the intraoral image processing apparatus 100 may include, after the operation of changing the inner shape of the prosthesis, an operation of simplifying and smoothing the changed inner shape of the prosthesis.

FIG. 9 is a diagram for describing a reference direction, a prosthesis, and an inner shape of the prosthesis according to an embodiment of the present disclosure.

Referring to FIG. 9, a prosthesis 3000 generated based on an intraoral image including a prosthesis target tooth obtained by the intraoral image processing apparatus 100 is illustrated. In an embodiment, the prosthesis 3000 may be formed based on the shape of the outer surface (eggshell) of the prosthesis target tooth.

In an embodiment, the inner shape 2000 of the prosthesis may be formed based on the shape of the prosthesis target tooth. In an embodiment, the inner shape 2000 of the prosthesis may be formed based on the shape of the prosthesis target tooth, and the prosthesis 3000 may have a shape offset from the inner shape 2000 of the prosthesis toward the outside of the prosthesis target tooth.

In an embodiment, the preset reference insertion direction 4000 may be a direction preset according to the shape of the prosthesis target tooth and/or the shape of adjacent teeth around the prosthesis target tooth and may represent a normal direction of the prosthesis target tooth.

The inner shape 2000 of the prosthesis may be formed by applying the preset reference insertion direction 4000 to the image of the prosthesis target tooth. In an embodiment, the intraoral image processing apparatus 100 may generate the inner shape 2000 of the prosthesis 3000 in order to be able to insert the prosthesis 3000 into the prosthesis target tooth in the reference insertion direction 4000.

FIG. 10A is a diagram for describing a set direction and a virtual inner shape according to an embodiment of the present disclosure. Hereinafter, the same reference numerals will be assigned to the same elements as those described in FIG. 9, and redundant descriptions thereof will be omitted for conciseness.

Referring to FIG. 10A, a prosthesis 3000, an inner shape 2000 of the prosthesis generated based on the reference insertion direction 4000, and an insertion direction 4100 of the prosthesis 3000 corresponding to the prosthesis target tooth set based on the shape of the prosthesis target tooth are illustrated. In an embodiment, the insertion direction 4100 of the prosthesis 3000 may be set according to the shape of the prosthesis target tooth and the shapes of adjacent teeth around the prosthesis target tooth. In an embodiment, the insertion direction 4100 of the prosthesis 3000 may be set according to an area included in the prosthesis target tooth to be dentally treated by using the prosthesis 3000.

In an embodiment, FIG. 10A illustrates a virtual inner surface shape 5100 generated by applying the insertion direction 4100 of the prosthesis 3000 to an intraoral image including the prosthesis target tooth. In an embodiment, a margin line 5000 of the inner shape 2000 of the prosthesis and a margin line 5000 of the virtual inner shape 5100 may be the same as each other. In an embodiment, the inner shape 2000 of the prosthesis may have a shape extending from the margin line 5000 in the reference insertion direction 4000. The virtual inner shape 5100 may have a shape extending from the margin line 5000 in the insertion direction 4100.

FIG. 10B is a diagram for describing an operation of changing an inner shape of a prosthesis according to an embodiment of the present disclosure. Hereinafter, for convenience of description, the same reference numerals will be assigned to the same elements as those described in FIG. 10A, and redundant descriptions thereof will be omitted for conciseness.

Referring to FIG. 10B, the inner shape 2000 of the prosthesis may be changed by comparing the inner shape 2000 of the prosthesis with the virtual inner shape 5100. In an embodiment, the inner shape 2000 of the prosthesis may include a plurality of points along the surface of the inner shape 2000. The at least one processor 150 may provide a virtual line in a normal direction toward the inside of the inner shape 2000 of the prosthesis from each of the plurality of points. The at least one processor 150 may define, as a correction area 2100, an area of the inner shape 2000 of the prosthesis including at least one point providing a virtual line intersecting the virtual inner shape 5100. The at least one processor 150 may change the shape of the correction area 2100 to correspond to the virtual inner shape 5100. In an embodiment, the at least one processor 150 may move the correction area 2100 toward the virtual inner shape 5100 or delete the correction area 2100 located outside the virtual inner shape 5100 to change the inner shape 2000 of the prosthesis to correspond to the virtual inner shape 5100.

In an embodiment, the prosthesis 3000 may be inserted into the prosthesis target tooth in the insertion direction 4100 different from the preset reference insertion direction 4000, according to the shape of the prosthesis target tooth or the arrangement of the prosthesis target tooth and adjacent teeth around the prosthesis target tooth. In this case, when the prosthesis 3000 having the inner shape 2000 of the prosthesis generated based on the preset reference insertion direction 4000 is inserted into the prosthesis target tooth in the insertion direction 4100, an undercut area 2100 may occur and thus it may not be easy to insert the prosthesis 3000 thereinto. Through the intraoral image processing apparatus and method according to the present disclosure, the inner shape 2000 of the prosthesis may be changed to correspond to the insertion direction 4100 and thus the prosthesis 3000 may be easily coupled to the prosthesis target tooth in the insertion direction 4100.

FIG. 10C is a diagram for describing an intraoral image processing apparatus and method according to an embodiment of the present disclosure.

Referring to FIGS. 10B and 10C, FIG. 10C illustrates an inner shape 2000-1 of the prosthesis changed from the inner shape 2000 of the prosthesis to correspond to the virtual inner shape 5100. In an embodiment, the changed inner shape 2000-1 of the prosthesis may be changed from the inner shape 2000 of the prosthesis to correspond to the set insertion direction 4100.

In an embodiment, when a pre-preparation prosthesis target tooth is prepared to have a shape corresponding to the changed inner shape 2000-1 of the prosthesis, the prosthesis 3000 illustrated in FIG. 10C may be inserted into a tooth prepared to have a shape corresponding to the changed inner shape 2000-1 of the prosthesis. Thus, the intraoral image processing apparatus 100 (see FIG. 1) and the intraoral image processing method of the present disclosure may set an insertion direction 1000 for preventing a dentally treated area from becoming an undercut area according to the position of an area requiring dental treatment and may change the inside of the prosthesis 3000 based on the set insertion direction 1000. Also, the dentally treated area may be prevented from becoming an undercut area, and the prosthesis 3000 may be easily inserted into the prepared tooth.

The intraoral image processing method according to an embodiment may be embodied in the form of program commands executable through various computer means and then may be recorded on a computer-readable recording medium. Also, an embodiment of the present disclosure may be a computer-readable storage medium having recorded thereon one or more programs including at least one instruction for executing the intraoral image processing method.

The computer-readable storage medium may include program instructions, data files, and data structures either alone or in combination. Here, examples of the computer-readable storage medium may include magnetic media such as hard disks, floppy disks, and magnetic tapes, optical media such as CD-ROMs and DVDs, magneto-optical media such as floptical disks, and hardware devices such as ROMs, RAMs, and flash memories that are configured to store and execute program instructions.

Here, a machine-readable storage medium may be provided in the form of a non-transitory storage medium. Here, the ‘non-transitory storage medium’ may mean that the storage medium is a tangible device. Also, the ‘non-transitory storage medium’ may include a buffer in which data is temporarily stored.

According to an embodiment, the intraoral image processing method according to various embodiments described herein may be included and provided in a computer program product. The computer program product may be distributed in the form of a machine-readable storage medium (e.g., a compact disc read only memory (CD-ROM)). Alternatively, the computer program product may be distributed (e.g., downloaded or uploaded) online through an application store (e.g., Play Store) or directly between two user devices (e.g., smart phones). Particularly, the computer program product according to the embodiments may include a storage medium having recorded thereon a program including at least one instruction for performing the intraoral image processing method according to the embodiments.

Although embodiments have been described above in detail, the scope of the present disclosure is not limited thereto and various modifications and improvements made by those of ordinary skill in the art by using the basic concept of the present disclosure defined in the following claims are also included in the scope of the present disclosure.

1. An intraoral image processing method comprising:
obtaining an intraoral image including a tooth;
setting an insertion direction of a prosthesis corresponding to the tooth;
obtaining an undercut area included in the tooth based on the set insertion direction and the intraoral image; and
compensating the undercut area based on the set insertion direction.

2. The intraoral image processing method of claim 1, wherein the tooth includes a plurality of points along a surface shape of the tooth,
the setting of the insertion direction comprises setting the insertion direction based on a shape of the tooth, and
the insertion direction is set based on at least one of the shape of the tooth, a shape of adjacent teeth around the tooth, an arrangement between the tooth and the adjacent teeth, or an average normal direction of the plurality of points included in the tooth.

3. The intraoral image processing method of claim 2, wherein the obtaining of the undercut area comprises:
providing a virtual line in a direction parallel to the insertion direction from each of the plurality of points; and
obtaining, as the undercut area, an area of the tooth including at least one point providing a virtual line intersecting the tooth.

4. The intraoral image processing method of claim 3, wherein, when the at least one point included in the undercut area is defined as a first reference point, at least one point meeting the virtual line provided from the first reference point is defined as a second reference point, and a center of an extension line passing through the first reference point and the second reference point is defined as a third reference point, the compensating of the undercut area comprises:
obtaining a center point located at a center of the tooth;
obtaining a reference direction from the center point toward the third reference point; and
moving the first reference point in a direction parallel to the reference direction to compensate the undercut area.

5. The intraoral image processing method of claim 4, wherein the compensating of the undercut area further comprises, before the moving of the first reference point, moving the center point to be on a same line as the first reference point.

6. The intraoral image processing method of claim 4, wherein the compensating of the undercut area further comprises:
obtaining a first reference direction from the center point toward the first reference point;
calculating a normal direction from the first reference point toward an outside of the tooth; and
when an angle between a vector with the first reference direction and a vector with the normal direction is greater than 90 degrees, moving the first reference point in a direction opposite to the first reference direction to compensate the undercut area.

7. The intraoral image processing method of claim 3, wherein the compensating of the undercut area further comprises moving at least one point included in the undercut area in a direction perpendicular to the insertion direction and toward an outside of the tooth, to compensate the undercut area.

8. The intraoral image processing method of claim 3, wherein the compensating of the undercut area is repeated until the virtual line provided from each of the plurality of points does not intersect the tooth in the obtaining of the undercut area.

9. The intraoral image processing method of claim 1, further comprising, after the compensating of the undercut area, simplifying and smoothing the compensated undercut area.

10. An intraoral image processing apparatus comprising:
a memory storing at least one instruction; and
at least one processor configured to execute the at least one instruction stored in the memory,
wherein the at least one processor is configured to
obtain an intraoral image including a tooth,
set an insertion direction of a prosthesis corresponding to the tooth,
obtain an undercut area included in the tooth based on the set insertion direction and the intraoral image, and
compensate the undercut area based on the set insertion direction.

11. The intraoral image processing apparatus of claim 10, wherein the at least one processor is configured to
define a plurality of points in the tooth along a surface shape of the tooth, and
set the insertion direction based on a shape of the tooth,
wherein the insertion direction is set based on at least one of the shape of the tooth, a shape of adjacent teeth around the tooth, an arrangement between the tooth and the adjacent teeth, or an average normal direction of the plurality of points included in the tooth.

12.-15. (canceled)

16. The intraoral image processing apparatus of claim 11, wherein the at least one processor is configured to
provide a virtual line in a direction parallel to the insertion direction from each of the plurality of points, and
obtain, as the undercut area, an area of the tooth including at least one point providing a virtual line intersecting the tooth.

17. The intraoral image processing apparatus of claim 16, wherein, when the at least one point included in the undercut area is defined as a first reference point, at least one point meeting the virtual line provided from the first reference point is defined as a second reference point, and a center of an extension line passing through the first reference point and the second reference point is defined as a third reference point, the at least one processor is configured to
obtain a center point located at a center of the tooth,
obtain a reference direction from the center point toward the third reference point, and
move the first reference point in a direction parallel to the reference direction to compensate the undercut area.

18. The intraoral image processing apparatus of claim 17, wherein the at least one processor is configured to
before the moving of the first reference point, move the center point to be on a same line as the first reference point.

19. The intraoral image processing apparatus of claim 17, wherein the at least one processor is configured to
obtain a first reference direction from the center point toward the first reference point,
calculate a normal direction from the first reference point toward an outside of the tooth, and
when an angle between a vector with the first reference direction and a vector with the normal direction is greater than 90 degrees, move the first reference point in a direction opposite to the first reference direction to compensate the undercut area.

20. The intraoral image processing apparatus of claim 16, wherein the at least one processor is configured to
move at least one point included in the undercut area in a direction perpendicular to the insertion direction and toward an outside of the tooth, to compensate the undercut area.

21. The intraoral image processing apparatus of claim 16, wherein the at least one processor is configured to
repeat the compensating of the undercut area until the virtual line provided from each of the plurality of points does not intersect the tooth in the obtaining of the undercut area.

22. The intraoral image processing apparatus of claim 10, wherein the at least one processor is configured to
after the compensating of the undercut area, simplify and smooth the compensated undercut area.

23. A non-transitory computer-readable recording medium having recorded thereon a program which, when executed by at least one processor of a computer, causes the computer to:
obtain an intraoral image including a tooth,
set an insertion direction of a prosthesis corresponding to the tooth,
obtain an undercut area included in the tooth based on the set insertion direction and the intraoral image, and
compensate the undercut area based on the set insertion direction.
